(19) United States
(12) Patent Application Publication
(10) Pub. No.: US 2006/0189613 A1
(43) Pub. Date: Aug. 24, 2006

(54) Sulphonamide Compounds that Modulate Chemokine Receptor Activity (CCR4)

(76) Inventor: David CHESHIRE, Leicestershire (GB)

Correspondence Address: FISH & RICHARDSON P.C., P.O BOX 1022, MINNEAPOLIS, MN, 55440-1022, US

(21) Appl. No.: 10/559,202

(22) PCT Filed: Jun. 2, 2004
(86) PCT No.: PCT/SE04/00851

§ 371 Date: Dec. 2, 2005

(30) Foreign Application Priority Data
Jun. 5, 2003 (SE) 0301654-0

(51) Int. Cl. A61K 31/53; A61K 31/513; C07D 405/02

(52) U.S. Cl. 514/241; 514/269; 544/209; 544/314

(57) ABSTRACT

The invention provides pyrimidine, pyridazine and triazine compounds for use in therapy.

The present invention relates to sulphonamide compounds, processes and intermediates used in their preparation, pharmaceutical compositions containing them and their use in therapy.

Chemokines play an important role in immune and inflammatory responses in various diseases and disorders, including asthma and allergic diseases, as well as autoimmune pathologies such as rheumatoid arthritis and atherosclerosis. These small-secreted molecules are a growing superfamily of 8-14 kDa proteins characterised by a conserved four cysteine motif. At the present time, the chemokine superfamily comprises three groups exhibiting characteristic structural motifs, the Cys-X-Cys (C—X—C), Cys-Cys (C—C) and Cys-X₃-Cys (C—X₃—C) families. The C—X—C and C—C families have sequence similarity and are distinguished from one another on the basis of a single amino acid insertion between the NH-proximal pair of cysteine residues. The C—X₃—C family is distinguished from the other two families on the basis of having a triple amino acid insertion between the NH-proximal pair of cysteine residues.

The C—X—C chemokines include several potent chemoattractants and activators of neutrophils such as interleukin-8 (IL-8) and neutrophil-activating peptide 2 (NAP-2).

The C—C chemokines include potent chemoattractants of monocytes and lymphocytes but not neutrophils. Examples include human monocyte chemotactic proteins 1-3 (MCP-1, MCP-2 and MCP-3), RANTES (Regulated on Activation, Normal T Expressed and Secreted), eotaxin and the macrophage inflammatory proteins 1α and 1β (MIP-1α and MIP-1β), Thymus and Activation Regulated Chemokine (TARC, CCL17) and Macrophage Derived Chemokine (MDC, CCL22). The C—X₃—C chemokine (also known as fractalkine) is a potent chemoattractant and activator of microglia in the central nervous system (CNS) as well as of monocytes, T cells, NK cells and mast cells.

Studies have demonstrated that the actions of chemokines are mediated by subfamilies of G protein-coupled receptors, among which are the receptors designated CCR1, CCR2, CCR2A, CCR2B, CCR3, CCR4, CCR5, CCR6, CCR7, CCR8, CCR9, CCR10 and CCR11 (for the C—C family); CXCR1, CXCR2, CXCR3, CXCR4 and CXCR5 (for the C—X—C family) and CX₃CR1 for the C—X₃—C family. These receptors represent good targets for drug development since agents which modulate these receptors would be useful in the treatment of disorders and diseases such as those mentioned above.

WO03/05 1870 and WO03/059893 disclose a series of sulphonamide compounds said to be useful for treating various diseases. It has now surprisingly been found that a series of diazines and triazines are active at the CCR4 receptor.

The present invention therefore provides a compound of formula (1) and pharmaceutically acceptable salts or solvates thereof:

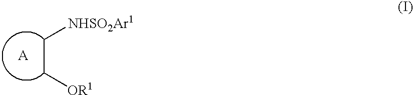

in which:

Ar¹ is dichlorophenyl or thienyl substituted by one or two chlorine atoms;

A is a pyrimidine, pyridazine or 1,2,4-triazine ring, each of which can be optionally substituted by one or more groups selected from hydroxyl, halogen, cyano, C₁₋₆alkyl, C₁₋₆alkoxy or C₃₋₆cycloalkyl where in each case the alkyl group may be substituted with 1-3 fluorine atoms, a cyano group or a hydroxy group;

R¹ is C₁₋₆alkyl or C₃₋₆cycloalkyl each of which can be optionally substituted with 1-3 fluorine atoms or a cyano group or R¹ is C₃₋₆alkenyl or C₃₋₆alkynyl or C₁₋₆alkyl-R²

R² is an aryl group or a 5-7 membered heteroaromatic ring containing 1-4 heteroatoms selected from nitrogen, oxygen or sulphur each of which can be optionally substituted by 1-3 groups selected from halogen, C₁₋₆alkyl, C₁₋₆alkoxy, ═O, ═S, CN or (CH₂)nOH where n is 1 or 2.

Certain compounds of formula (I) are capable of existing in stereoisomeric forms. It will be understood that the invention encompasses all geometric and optical isomers of the compounds of formula (I) and mixtures thereof including racemates. Tautomers and mixtures thereof also form an aspect of the present invention.

Preferably Ar¹ is 2,3-dichlorophenyl.

Preferably A is Pyrimidine. Preferred substituents for the ring A include halogen, in particular chloro, and C₁₋₆alkoxy, in particular methoxy.

Preferably R¹ is C₁₋₆alkyl, in particular methyl.

Preferably, where Ar¹ is dichlorophenyl, A is pyrimidine or a 1,2,4-triazine ring. Preferred compounds of formula (I) include:

2,3-Dichloro-N-[4-methoxy-3-pyridazinyl]benzenesulphonamide

2,3-Dichloro-N-[6-chloro-4-methoxy-3-pyridazinyl]benzenesulphonamide.

2,3-Dichloro-N-[6-chloro-4-(3-pyridinylmethoxy)-3-pyridazinyl]benzenesulphonamide.

2,3-Dichloro-N-[3-chloro-6-methoxy-1,2,4-triazin-5-yl]benzenesulphonamide

2,3-Dichloro-N-[2,4-dimethoxy-5-pyrimidinyl]benzenesulphonamide

2,3-Dichloro-N-[4-methoxy-5-pyrimidinyl]benzenesulphonamide

2,3-Dichloro-N-[2-chloro-5-methoxy-4-pyrimidinyl]benzenesulphonamide

2,3-Dichloro-N-[5-methoxy-4-pyrimidinyl]benzenesulphonamide

2,3-Dichloro-N-[5-methoxy-2-methyl-4-pyrimidinyl]benzenesulphonamide

2,3-Dichloro-N-[5-methoxy-2-trifluoromethyl-4-pyrimidinyl]benzenesulphonamide

5-Chloro-thiophene-2-sulphonic acid, [2-chloro-5-methoxy-4-pyrimidinyl]amide

5-Chloro-thiophene-2-sulphonic acid, [5-methoxy-2-methyl-4-pyrimidinyl]amide

5-Chloro-N-[6-chloro-4-methoxy-3-pyridazinyl]thiophene-2-sulphonamide

and pharmaceutically acceptable salts and solvates thereof.

Compounds of the invention can be prepared using processes known in the art, for example by reacting a compound of formula (II):

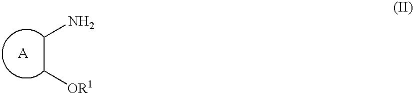

in which A and R¹ are as defined in formula (I) or are protected derivatives thereof, with a compound of formula (III):
Ar¹SO₂L (III)

in which Ar¹ is as defined in formula (I) or is a protected derivative thereof, and L is a leaving group,

and optionally thereafter:
removing any protecting groups
forming a pharmaceutically acceoptable salt or solvate.

The leaving group L may conveniently be a halogen, for example chloro or bromo. The reaction can be carried out in the presence of a base, e.g. potassium t-butoxide, and performed in a solvent, e.g. THF. Compounds of formula (II) and (III) can be prepared using standard chemistry.

It will be appreciated by those skilled in the art that in the processes of the present invention certain functional groups in the starting reagents or intermediate compound may need to be protected by protecting groups. Thus, the preparation of the compound of formula (I) may involve, at an appropriate stage, the removal of one or more protecting groups. The protection and deprotection of functional groups is fully described in ‘Protective Groups in Organic Chemistry’, edited by J. W. F. McOmie, Plenum Press (1973), and ‘Protective Groups in Organic Synthesis’, 3rd edition, T. W. Greene & P. G. M. Wuts, Wiley-Interscience (1999).

The compounds of formula (I) above may be converted to a pharmaceutically acceptable salt or solvate thereof, preferably a basic addition salt such as sodium, potassium, calcium, aluminium, lithium, magnesium, zinc, benzathine, chloroprocaine, choline, diethanolamine, ethanolamine, ethyldiamine, meglumine, tromethamine or procaine, or an acid addition salt such as a hydrochloride, hydrobromide, phosphate, acetate, fumarate, maleate, tartrate, citrate, oxalate, methanesulphonate or p-toluenesulphonate.

The compounds of formula (1) has activity as pharmaceuticals, in particular as modulators of chemokine receptor (especially CCR4) activity, and may be used in the treatment (therapeutic or prophylactic) of conditions/diseases in human and non-human animals which are exacerbated or caused by excessive or unregulated production of chemokines. Examples of such conditions/diseases include:
(1) (the respiratory tract) obstructive airways diseases including chronic obstructive pulmonary disease (COPD); asthma, such as bronchial, allergic, intrinsic, extrinsic and dust asthma, particularly chronic or inveterate asthma (e.g. late asthma and airways hyper-responsiveness); bronchitis; acute, allergic, atrophic rhinitis and chronic rhinitis including rhinitis caseosa, hypertrophic rhinitis, rhinitis purulenta, rhinitis sicca and rhinitis medicamentosa; membranous rhinitis including croupous, fibrinous and pseudomembranous rhinitis and scrofoulous rhinitis; seasonal rhinitis including rhinitis nervosa (hay fever) and vasomotor rhinitis; sarcoidosis, farmer's lung and related diseases, fibroid lung and idiopathic interstitial pneumonia;
(2) (bone and joints) gout, rheumatoid arthritis, seronegative spondyloarthropathies (including ankylosing spondylitis, psoriatic arthritis and Reiter's disease), Behcet's disease, Sjogren's syndrome and systemic sclerosis;
(3) (skin) pruritis, scleroderma, otitus, psoriasis, atopical dermatitis, contact dermatitis and other eczmatous dermitides, seborrhoetic dermatitis, Lichen planus, Pemphigus, bullous Pemphigus, Epidermolysis bullosa, urticaria, angiodermas, vasculitides, erythemas, cutaneous eosinophilias, uveitis, Alopecia areata and vernal conjunctivitis, lupus;

(4) (gastrointestinal tract) Coeliac disease, proctitis, eosinopilic gastro-enteritis, mastocytosis, inflammatory bowel diseases such as Crohn's disease, ulcerative colitis, ileitis and enteritis, food-related allergies which have effects remote from the gut, e.g., migraine, rhinitis and eczema;

(5) (central and peripheral nervous system) Neurodegenerative diseases and dementia disorders, e.g. Alzheimer's disease, amyotrophic lateral sclerosis and other motor neuron diseases, Creutzfeldt-Jacob's disease and other prion diseases, HIV encephalopathy (AIDS dementia complex), Huntington's disease, frontotemporal dementia, Lewy body dementia and vascular dementia; polyneuropathies, e.g. Guillain-Barré syndrome, chronic inflammatory demyelinating polyradiculoneuropathy, multifocal motor neuropathy, plexopathies; CNS demyelination, e.g. multiple sclerosis, acute disseminated/haemorrhagic encephalomyelitis, and subacute sclerosing panencephalitis; neuromuscular disorders, e.g. myasthenia gravis and Lambert-Eaton syndrome; spinal diorders, e.g. tropical spastic paraparesis, and stiff-man syndrome: paraneoplastic syndromes, e.g. cerebellar degeneration and encephalomyelitis; CNS trauma; migraine; stroke and correctum diseases such as meningitis
(6) (other tissues and systemic disease) hepatitis, vasculitis, spondyloarthopathies, vaginitis, glomerulonephritis, myositis, atherosclerosis, Acquired Immunodeficiency Syndrome (AIDS), lupus erythematosus, systemic lupus, erythematosus, Hashimoto's thyroiditis, type I diabetes, nephrotic syndrome, eosinophilia fascitis, hyper IgE syndrome, lepromatous leprosy, and idiopathic thrombocytopenia pupura; post-operative adhesions, and sepsis.
(7) (allograft and xenograft rejection) acute and chronic following, for example, transplantation of kidney, heart, liver, lung, bone marrow, skin and cornea; and chronic graft versus host disease;
(8) Cancer, carcinoma & tumour metastasis, including that of the bladder, breast, colon, kidney, liver, lung, ovary, pancreas, stomach, cervix, thyroid and skin, especially non-small cell lung cancer (NSCLC), malignant melanoma, prostate cancer and squamous sarcoma. Hematopoietic tumors of lymphoid lineage, including acute lymphocytic leukemia, B cell lymphoma and Burketts lymphoma, Hodgkins Lymphoma, Acute Lymphoblastic Leukemia. Hematopoietic tumors of myeloid lineage, including acute and chronic myelogenous leukemias and promyelocytic leukemia. Tumors of mesenchymal origin, including fibrosarcoma and rhabdomyosarcoma, and other tumors, including melanoma, seminoma, tetratocarcinoma, neuroblastoma and glioma.
(9) All diseases that result from a general inbalance of the immune system and resulting in increased atopic inflammatory reactions.
(10) Cystic fibrosis, re-perfusion injury in the heart, brain, peripheral limbs and other organs.
(11) Burn wounds & chronic skin ulcers
(12) Reproductive Diseases (e.g. Disorders of ovulation, menstruation and implantation, Pre-term labour, Endometriosis)
(13) thrombosis
(14) infectious diseases such as HIV infection and other viral infections, bacterial infections.

Thus, the present invention provides a compound of formula (I), or a pharmaceutically-acceptable salt or solvate thereof, as hereinbefore defined for use in therapy.

Preferably the compound of the invention are used to treat diseases in which the chemokine receptor belongs to the CC chemokine receptor subfamily, more preferably the target chemokine receptor is the CCR4 receptor.

Particular conditions which can be treated with the compound of the invention are asthma, rhinitis and inflammatory skin disorders, diseases in which there are raised TARC, MDC or CCR4 levels. It is preferred that the compound of the invention is used to treat asthma and rhinitis, especially asthma.

In a further aspect, the present invention provides the use of a compound of formula (I), or a pharmaceutically acceptable salt or solvate thereof, as hereinbefore defined in the manufacture of a medicament for use in therapy.

In a still further aspect, the present invention provides the use of a compound of formula (I), or a pharmaceutically acceptable salt or solvate thereof, as hereinbefore defined in the manufacture of a medicament for the treatment of human diseases or conditions in which modulation of chemokine receptor activity, particularly CCR4 activity, is beneficial.

In a further aspect, the present invention provides the use of a compound or formula (I), or a pharmaceutically acceptable salt or solvate thereof, as hereinbefore defined in combination with other drugs used to treat asthma and rhinitis (such as inhaled and oral steroids, inhaled β2-receptor agonists and oral leukotriene receptor antagonists and the like).

In the context of the present specification, the term “therapy” also includes “prophylaxis” unless there are specific indications to the contrary. The terms “therapeutic” and “therapeutically” should be construed accordingly.

The invention still further provides a method of treating a chemokine mediated disease wherein the chemokine binds to a chemokine (especially CCR4) receptor, which comprises administering to a patient a therapeutically effective amount of a compound of formula (I), or a pharmaceutically acceptable salt or solvate thereof, as hereinbefore defined.

The invention also provides a method of treating a respiratory disease, such as asthma and rhinitis, especially asthma, in a patient suffering from, or at risk of, said disease, which comprises administering to the patient a therapeutically effective amount of a compound of formula (I), or a pharmaceutically acceptable salt or solvate thereof, as hereinbefore defined.

For the above-mentioned therapeutic uses the dosage administered will, of course, vary with the compound employed, the mode of administration, the treatment desired and the disorder indicated.

The compound of formula (I) and pharmaceutically acceptable salts and solvates thereof may be used on their own but will generally be administered in the form of a pharmaceutical composition in which the formula (I) compound/salt/solvate (active ingredient) is in association with a pharmaceutically acceptable adjuvant, diluent or carrier. Depending on the mode of administration, the pharmaceutical composition will preferably comprise from 0.05 to 99% w (per cent by weight), more preferably from 0.05 to 80% w, still more preferably from 0.10 to 70% w, and even more preferably from 0.10 to 50% w, of active ingredient, all percentages by weight being based on total composition.

The present invention also provides a pharmaceutical composition comprising a compound of formula (I), or a pharmaceutically acceptable salt or solvate thereof, as hereinbefore defined, in association with a pharmaceutically acceptable adjuvant, diluent or carrier.

The invention further provides a process for the preparation of a pharmaceutical composition of the invention which comprises mixing a compound of formula (I), or a pharmaceutically acceptable salt or solvate thereof, as hereinbefore defined, with a pharmaceutically acceptable adjuvant, diluent or carrier.

The pharmaceutical compositions may be administered topically (e.g. to the lung and/or airways or to the skin) in the form of solutions, suspensions, heptafluoroalkane aerosols and dry powder formulations; or systemically, e.g. by oral administration in the form of tablets, capsules, syrups, powders or granules, or by parenteral administration in the form of solutions or suspensions, or by subcutaneous administration or by rectal administration in the form of suppositories or transdermally. Preferably the compound of the invention is administered orally.

The invention further relates to combination therapies for the treatment of any one of rheumatoid arthritis, osteoarthritis, osteoporosis, psoriasis, inflammatory bowel diseases, COPD asthma, allergic rhinitis, atopic dermatitis or cancer or the neurodegenerative diseases such as multiple sclerosis, Alzheimer's disease or stroke.

For the treatment of rheumatoid arthritis, the compounds of the invention may be combined with “biological agents” such as TNF-α inhibitors such as anti-TNF monoclonal antibodies (such as Remicade, CDP-870 and Humira) and soluble TNF receptor immunoglobulin molecules (such as Enbrel.reg.). IL-1 receptor antagonist (such as Anakinra) and IL-1 trap, IL-18 receptor, anti-IL-6 Ab, anti-CD20 Ab, anti-IL-15 Ab and CTLA4Ig.

Suitable agents to be used in combination include standard non-steroidal anti-inflammatory agents (hereinafter NSAID's) such as piroxicam, diclofenac, propionic acids such as naproxen, flubiprofen, fenoprofen, ketoprofen and ibuprofen, fenamates such as lo mefenamic acid, indomethacin, sulindac, apazone, pyrazolones such as phenylbutazone, salicylates such as aspirin. The cyclooxygenase-2 (COX-2) inhibitors (such as meloxicam, celecoxib, rofecoxib, valdecoxib and etoricoxib) and the cyclo-oxygenase inhibiting nitric oxide donors (CINOD's) and the “disease modifying agents” (DMARDs) such as methotrexate, sulphasalazine, cyclosporine A, lefunomide; ciclesonide; hydroxychloroquine, d-penicillamine, auranofin or parenteral or oral gold.

The present invention still further relates to the combination of a compound of the invention together with a leukotriene biosynthesis inhibitor, 5-lipoxygenase (5-LO) inhibitor or 5-lipoxygenase activating protein (FLAP) antagonist selected from the group consisting of zileuton; ABT-761; fenleuton; tepoxalin; Abbott-79175; Abbott-85761; N-(5-substituted)-thiophene-2-alkylsulfonamides; 2,6-di-tert-butylphenol hydrazones; methoxytetrahydropyrans such as Zeneca ZD-2138; the compound SB-210661; pyridinyl-substituted 2n cyanonaphthalene compounds such as L-739,010; 2-cyanoquinoline compounds such as L-746,530; indole and quinoline compounds such as MK-591, MK-886, and BAY×1005.

The present invention still further relates to the combination of a compound of the invention together with a receptor antagonists for leukotrienes LTB₄, LTC₄, LTD₄, and LTE₄ selected from the group consisting of the phenothiazin-3-ones such as L-651,392; amidino compounds such as CGS-25019c; benzoxalamines such as ontazolast; benzenecarboximidamides such as BIIL 284/260; and compounds such as zafirlukast, ablukast, montelukast, pranlukast, verlukast (MK-679), RG-12525, Ro-245913, iralukast (CGP 45715A), and BAY×7195.

The present invention still further relates to the combination of a compound of the invention together with a phosphodiesterase-4 (PDE4) inhibitor including inhibitors of the isoform PDE4D.

The present invention still further relates to the combination of a compound of the invention together with histaminic H₁ receptor antagonists including cetirizine, loratadine, desloratadine, fexofenadine, astemizole, azelastine, and chlorpheniramine.

The present invention still further relates to the combination of a compound of the invention together with a gastroprotective histaminic H₂ receptor antagonist or the proton pump inhibitors (such as omeprazole)

The present invention still further relates to the combination of a compound of the invention together with an α₁- and α₂-adrenoceptor agonist vasoconstrictor sympathomimetic agent, including propylhexedrine, phenylephrine, phenylpropanolamine, pseudoephedrine, naphazoline hydrochloride, oxymetazoline hydrochloride, tetrahydrozoline hydrochloride, xylometazoline hydrochloride, and ethylnorepinephrine hydrochloride.

The present invention still further relates to the combination of a compound of the invention together with anticholinergic agents including ipratropium bromide; tiotropium bromide; oxitropium bromide; pirenzepine; and telenzepine.

The present invention still further relates to the combination of a compound of the invention together with a β₁- to β₄-adrenoceptor agonists including metaproterenol isoproterenol, isoprenaline, albuterol, salbutamol, formoterol, salmeterol, terbutaline, orciprenaline, bitolterol mesylate, and pirbuterol; or methylxanthanines including theophylline and aminophylline; sodium cromoglycate; or muscarinic receptor (M1, M2, and M3) antagonist.

The present invention still further relates to the combination of a compound of the invention together with other modulators of chemokine receptor function such as CCR1, CCR2, CCR2A, CCR2B, CCR3, CCR5, CCR6, CCR7, CCR8, CCR9, CCR10 and CCR11 (for the C—C family); CXCR1, CXCR2, CXCR3, CXCR4 and CXCR5 (for the C—X—C family) and CX₃CR1 for the C—X₃—C family.

The present invention still further relates to the combination of a compound of the invention together with an insulin-like growth factor type I (IGF-1) mimetic.

The present invention still further relates to the combination of compound of the invention together with an inhaled glucocorticoid with reduced systemic side effects, including prednisone, prednisolone, flunisolide, triamcinolone acetonide, beclomethasone dipropionate, budesonide, fluticasone propionate, and mometasone furoate.

The present invention still further relates to the combination of a compound of the invention together with (a) tryptase inhibitors; (b) platelet activating factor (PAF) antagonists; (c) interleukin converting enzyme (ICE) inhibitors; (d) IMPDH inhibitors; (e) adhesion molecule inhibitors including VLA-4 antagonists; (f) cathepsins; (g) MAP kinase inhibitors; (h) glucose-6 phosphate dehydrogenase inhibitors; (i) kinin-B₁- and B₂-receptor antagonists; (j) anti-gout agents, e.g., colchicine; (k) xanthine oxidase inhibitors, e.g., allopurinol; (l) uricosuric agents, e.g., probenecid, sulfinpyrazone, and benzbromarone; (m) growth hormone secretagogues; (n) transforming growth factor (TGFβ); (o) platelet-derived growth factor (PDGF); (p) fibroblast growth factor, e.g., basic fibroblast growth factor (bFGF); (q) granulocyte macrophage colony stimulating factor (GM-CSF); (r) capsaicin cream; (s) Tachykinin NK1 and NK3 receptor antagonists selected from the group consisting of NKP-608C; SB-233412 (talnetant); and D-4418; and (t) elastase inhibitors selected from the group consisting of UT-77 and ZD-0892 (u) induced nitric oxide synthase inhibitors (iNOS) or (v) chemoattractant receptor-homologous molecule expressed on TH2 cells, (CRTH2 antagonists).

The present invention still further relates to the combination of a compound of the invention together with an inhibitor of matrix metalloproteases (MMPs), i.e., the stromelysins, the collagenases, and the gelatinases, as well as aggrecanase; especially collagenase-1 (MMP-1), collagenase-2 (MMP-8), collagenase-3 (MMP-13), stromelysin-1 (MMP-3), stromelysin-2 (MMP-10), stromelysin-3 (MMP-11), and MP12 inhibitors.

The compounds of the invention can also be used in combination with existing therapeutic agents for the treatment of osteoarthritis. Suitable agents to be used in combination include standard non-steroidal anti-inflammatory agents (hereinafter NSAID's) such as piroxicam, diclofenac, propionic acids such as naproxen, flubiprofen, fenoprofen, ketoprofen and ibuprofen, fenamates such as mefenamic acid, indomethacin, sulindac, apazone, pyrazolones such as phenylbutazone, salicylates such as aspirin, induced nitric oxide synthase inhibitors (iNOS inhibitors), COX-2 inhibitors such as celecoxib, valdecoxib, rofecoxib and etoricoxib, and the cyclo-oxygenase inhibiting nitric oxide donors (CINOD's) analgesics (such as paracetamol and tramadol), cartilage sparing agents such as diacerein, doxycyline and glucosamine, and intra-articular therapies such as corticosteroids and hyaluronic acids such as hyalgan and synvisc and P2X7 antagonists.

The compounds of the invention can also be used in combination with existing therapeutic agents for the treatment of inflammatory bowel diseases (Ulcerative colitis and Crohn's disease). Suitable agents to be used include sulphasalazine, 5-amino-salicylates, the thiopurines, azathioprine and 6-mecaptorurine and corticosteroids such as budesonide.

The compounds of the invention may also be used in combination with antiviral agents such as Viracept, AZT, aciclovir and famciclovir, and antisepsis compounds such as Valant.

The compounds of the present invention may also be used in combination with cardiovascular agents such as calcium channel blockers, lipid lowering agents such as statins, fibrates, beta-blockers, angiotensin converting enzyme (ACE) inhibitors, Angiotensin-2 receptor antagonists and platelet aggregation inhibitors.

The compounds of the present invention may also be used in combination with CNS agents such as antidepressants (such as sertraline), anti-Parkinsonian drugs (such as deprenyl, L-dopa, Requip, Mirapex, MAOB inhibitors such as selegine and rasagiline, comP inhibitors such as Tasmar, A-2 inhibitors, dopamine reuptake inhibitors, NMDA antagonists, Nicotine agonists, Dopamine agonists and inhibitors of neuronal nitric oxide synthase), and anti Alzheimer's drugs such as donepezil, tacrine, COX-2 inhibitors, propentofylline or metryfonate.

The compounds of the present invention may also be used in combination with osteoporosis agents such as roloxifene, droloxifene, lasofoxifene or fosomax and immunosuppressant agents such as FK-506, rapamycin, cyclosporine, azathioprine, and methotrexate.

The compounds of the invention can also be used in combination with existing therapeutic agents for the treatment of cancer. Suitable agents to be used in combination include:
(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulphan and nitrosoureas); antimetabolites (for example antifolates such as fluoropyrimidines like 5-fluorouracil and tegafur, raltitrexed, methotrexate, cytosine arabinoside, hydroxyurea, gemcitabine and paclitaxel (Taxol®); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere); and topoisomerase inhibitors (for example epipodophyllotoxins like etoposide and teniposide, amsacrine, topotecan and camptothecin);
(ii) cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5a-reductase such as finasteride;
(iii) Agents which inhibit cancer cell invasion (for example metalloproteinase inhibitors like marimastat and inhibitors of urokinase plasminogen activator receptor function);
(iv) inhibitors of growth factor function, for example such inhibitors include growth factor antibodies, growth factor receptor antibodies (for example the anti-erbb2 antibody trastuzumab [Herceptin™] and the anti-erbb1 antibody cetuximab [C225]), farnesyl transferase inhibitors, tyrosine kinase inhibitors and serine/threonine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033)), for example inhibitors of the platelet-derived growth factor family and for example inhibitors of the hepatocyte growth factor family;
(v) antiangiogenic agents such as those which inhibit the effects of vascular endothelial growth factor, (for example the anti-vascular endothelial cell growth factor antibody bevacizumab [Avastin™], compounds such as those disclosed in International Patent Applications WO 97/22596, WO 97/30035, WO 97/32856 and WO 98/13354) and compounds that work by other mechanisms (for example linomide, inhibitors of integrin αvβ3 function and angiostatin);
(vi) vascular damaging agents such as Combretastatin A4 and compounds disclosed in International Patent Applications WO99/02166, WO00/40529, WO00/41669, WO01/92224, WO02/04434 and WO02/08213;
(vii) antisense therapies, for example those which are directed to the targets listed above, such as ISIS 2503, an anti-ras antisense;
(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

The following examples illustrate the invention.

### EXAMPLE 1

### 2,3-Dichloro-N-[4-methoxy-3-pyridazinyl]benzenesulphonamide

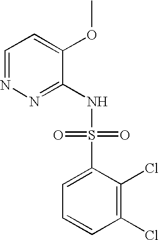

### a) 3-amino-6-chloro-4-methoxypyridazine

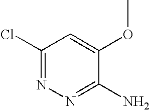

To a solution of 3-amino-4-bromo-6-chloropyridazine (2.08 g) in methanol (50 mL) was added sodium methoxide (6.5 mL of a 25 wt. % solution in methanol). The reaction mixture was stirred for 16 hours then treated with acetic acid (1.8 g). The reaction mixture was concentrated and the residue purified by chromatography on silica gel eluting with ethyl acetate to give the sub-titled compound (2.1 g).

m/e 159 (M+1⁺, 100%)

### b) 2,3-Dichloro-N-[4-methoxy-3-pyridazinyl]benzenesulphonamide

A suspension of 2-amino-6-chloro-4-methoxypyridazine (0.47 g), triethylamine (1 mL) and 10% palladium on carbon (0.1 g) in ethanol (15 mL) was hydrogenated at 1.2 atmospheres pressure until uptake of hydrogen was complete. The reaction mixture was filtered and the filtrate was concentrated to afford 3-amino-4-methoxypyridazine. This was dissolved in tetrahydrofuran (20 mL) and to the solution was added 2,3-dichlorobenzenesulphonyl chloride (0.87 g). Potassium tert-butoxide (12 mL of a 1M solution in tetrahydrofuran) was then added dropwise at 0° C. The reaction mixture was stirred at room temperature for 16 hours, diluted with ethyl acetate (100 mL) and washed with 1M aqueous hydrochloric acid solution (100 mL). The organic phase was dried Na₂SO₄), filtered and concentrated. The residue was purified by chromatography on silica gel eluting with tetrahydrofuran/iso-hexane mixtures and by recrystallisation from acetonitrile to give the title compound (0.014 g).

m/e 334 (M+1⁺, 100%)

¹H NMR (D6-DMSO) δ 8.41 (1H, br s), 8.07 (1H, d), 7.84 (1H, d), 7.52 (1H, t), 7.16 (1H, d), 3.89 (3H, s)

MP 210-211° C.

### EXAMPLE 2

### 2,3-Dichloro-N-[6-chloro-4-methoxy-3-pyridazinyl]benzenesulphonamide

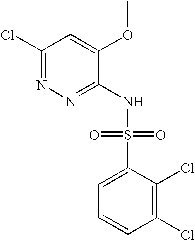

To a solution of 3-amino-6-chloro-4-methoxypyridazine (Example 1 part a) (0.5 g) in 1,2-dimethoxyethane (20 mL) was added 60% sodium hydride (0.25 g) in small portions. The mixture was stirred for 30 min then treated with 2,3-dichlorobenzenesulphonyl chloride (0.768 g) in one portion. The reaction mixture was stirred for 1 hour then acetic acid (0.384 g) was added. The mixture was concentrated and the residue purified by chromatography on silica gel eluting with ethyl acetate/iso-hexane mixtures to give the title compound (0.24 g) as a yellow powder.

m/e 370 (M+1⁺, 100%)

¹H NMR (D6-DMSO) δ 8.10 (1H, d), 7.95 (1H, d), 7.60 (1H, t), 7.43 (1H, s), 3.90 (3H, s)

MP 226-227° C.

### EXAMPLE 3

### 2,3-Dichloro-N-[6-chloro-4-(3-pyridinylmethoxy)-3-pyridazinyl]benzenesulphonamide

### a) 2,3-Dichloro-N-[6-chloro-4-bromo-3-pyridazinyl]benzenesulphonamide

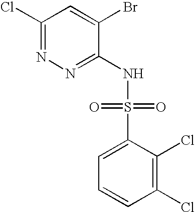

The sub-titled compound was prepared from 3-amino-4-bromo-6-chloropyridazine (0.5 g), 2,3-dichlorobenzenesulphonyl chloride (0.59 g) and 60% sodium hydride (0. 192 g) by the method of Example 1. The crude product was purified by chromatography on silica gel eluting with ethyl acetate/iso-hexane mixtures to give the sub-titled compound (0.64 g)

m/e 416 (M−1, 100%)

### b) 2,3-Dichloro-N-[6-chloro-4-(3-pyridinylmethoxy)-3-pyridazinyl]benzenesulphonamide

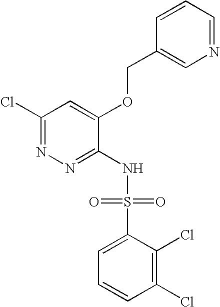

To a solution of 2,3-dichloro-N-[6-chloro-4-bromo-3-pyridazinyl]benzenesulphonamide (0.21 g) and pyridine-3-methanol (0.055 g) in 1,2-dimethoxyethane (10 mL) was added 60% sodium hydride (0.040 g) at 0° C. The reaction mixture was stirred at room temperature for 24 hours and treated with 5% aqueous citric acid (25 mL). The mixture was extracted with ethyl acetate and the combined organic extracts were dried (Na₂SO₄), filtered and concentrated. The residue was purified by chromatography on silica gel eluting with dichloromethane/iso-propanol mixtures and by trituration from diethyl ether to give the title compound (0.038 g).

m/e 445 (M+1⁺, 100%)

¹H NMR (D6-DMSO) δ 8.73 (1H, s), 8.61 (1H, d), 8.06 (1H, dd), 7.90 (2H, t), 7.6-7.45 (3H, m), 5.35 (2H, d)

MP 216-217° C.

### EXAMPLE 4

### 2,3-Dichloro-N-[3-chloro-6-methoxy-1,2,4-triazin-5-yl]benzenesulphonamide

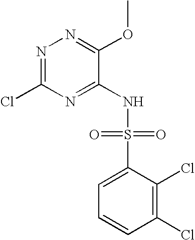

By the method outlined in Example 2 using 3-chloro-6-methoxy-1,2,4-triazin-5-amine (0.16 g), 2,3-dichlorobenzenesulphonyl chloride (0.27 g) and sodium hydride (60%, 0.1 g) in dry dimethoxyethane (10 mL) to afford the titled compound (0.132 g) as a solid after chromatography on silica gel eluting with ethyl acetate to 2% methanol in ethyl acetate.

m/e 368.8, 370.8 (M−1⁺, 100%)

¹H NMR (D6-DMSO) δ 8.01 (1H, dd), 7.73 (1H, dd), 7.46 (1H, dd), 3.83 (3H, s)

### EXAMPLE 5

### 2,3-Dichloro-N-[2,4-dimethoxy-5-pyrimidinyl]benzenesulphonamide

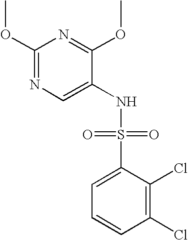

2,4-Dimethoxypyrimidin-5-amine (0.1 g) was dissolved in dry dichloromethane (5 mL) and pyridine (1 mL) and to this was added iso-butylchloroformate (0.11 mL). The resulting mixture was stirred at room temperature for 1 hour and was then poured into 1N hydrochloric acid and extracted into dichloromethane (2×10 mL), dried (MgSO₄), filtered and concentrated. The residue was dissolved in dry 1,2-dimethoxyethane (5 mL) and sodium hydride (60%, 0.032 g) was added, followed by 2,3-dichlorobenzenesulphonyl chloride (0.196 g). The reaction was stirred for 1 hour, poured into water and extracted into ethyl acetate (2×10 mL) and concentrated. The residue was dissolved in methanol (5 mL) and 1N sodium hydroxide (5 mL) added. The mixture was heated to reflux for 1 hour, cooled and concentrated to 5 mL, acidified by the addition of 2N hydrochloric acid and extracted into ethyl acetate, dried (MgSO₄), filtered and concentrated. The residue was purified by chromatography on silica gel eluting with ethyl acetate/iso-hexanes (1/2) to afford the titled compound as a solid.

m/e 364, 366 (M+1⁺, 100%)

¹H NMR (D6-DMSO) δ 10.1 (1H, s), 8.14 (1H, s), 7.94 (1H, dd), 7.77 (1H, dd), 7.47 (1H, dd), 3.85 (3H, s), 3.53 (3H, s).

M.P. 124-126° C.

### EXAMPLE 6

### 2,3-Dichloro-N-[4-methoxy-5-pyrimidinyl]benzenesulphonamide

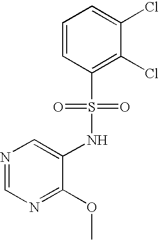

### a) 4-Methoxy-5-pyrimidinylamine

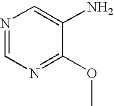

4-Chloro-6-methoxy-5-nitropyrimidine (0.6 g) and 5% Pd on carbon (0.6 g) in ethanol (90 mL) and under hydrogen (2 bar) was stirred at room temperature for 16 h. The mixture was filtered through a pad of celite and evaporated under reduced pressure to give the subtitle compound (0.35 g).

¹H NMR (D6-DMSO) δ 8.85 (1H, s), 7.97 (1H, s), 4.1 (3H, s)

### b) 2,3-Dichloro-N-[4-methoxy-5-pyrimidinyl]benzenesulphonamide

4-Methoxy-5-pyrimidinylamine (0.5 g) and 2,3-dichlorobenzenesulphonyl chloride (1.05 g) in pyridine (7 mL) was stirred at room temperature for 2 h. The pyridine was evaporated under reduced pressure then ethyl acetate (100 mL), methanol (10 mL) and acetic acid (1 mL) added. The solution was washed with water and brine and then evaporated under reduced pressure. Purification was by silica gel chromatography eluting with ethyl acetate/iso-hexanes (1/1) to give the title compound 0.35 g.

m/e 334 (M+1⁺, 100%)

¹H NMR (D6-DMSO) δ 10.52 (1H, br s), 8.61 (1H, s), 8.38 (1H, s), 7.95 (1H, dd), 7.84 (1H, dd), 7.51 (1H, t), 3.65 (3H, s)

MP 204-211° C.

### EXAMPLE 7

### 2,3-Dichloro-N-[2-chloro-5-methoxy-4-pyrimidinyl]benzenesulphonamide

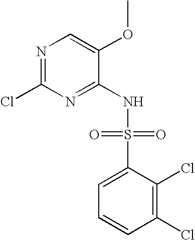

2,3-Dichloro-5-methoxy-pyrimidine(0.25 g), 2,3-dichlorobenzenesulphonamide(0.35 g), cesium carbonate(0.9 g) and dimethylformamide (10 mL) were heated together at 60° C. for six hours, allowed to cool, poured into water (100 mL), neutralised with dilute hydrochloric acid and extracted with ethylacetate, dried (MgSO₄) and evaporated under reduced pressure. Purification was by silica gel chromatography eluting with ethyl acetate/iso-hexane mixtures to give the above product (0.2 g)

m/e 366 (M−1⁺, 100%)

¹H NMR (DMSO-D6) δ 8.13-8.11 (1H, dd), 7.96 (1H, s), 7.87-7.85 (1H, dd), 7.55 (1H, t), 3.81 (3H, s)

MP 244-245° C.

### EXAMPLE 8

### 2,3-Dichloro-N-[5-methoxy-4-pyrimidinyl]benzenesulphonamide

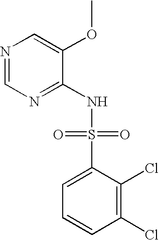

Prepared using the method of example 7 from 4-chloro-5-methoxypyrimidine.

m/e 334 (M+1⁺, 100%)

MP 278-279° C.

¹H NMR (D6-DMSO) δ 8.02-7.99 (1H, dd), 7.79 (1H, s), 7.66 (1H, dd), 7.61 (s, 1H), 7.39 (1H, t ), 3.72 (3H, s).

### EXAMPLE 9

### 2,3-Dichloro-N-[5-methoxy-2-methyl-4-pyrimidinyl]benzenesulphonamide

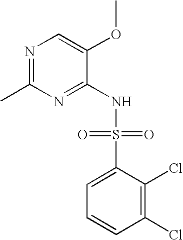

Prepared using the method of example 7 from 4-chloro-5-methoxy-2-methylpyrimidine

MP 234-235° C.

¹H NMR (D6-DMSO) δ 8.10-8.08 (1H, dd), 7.77-7.75 (1H, dd), 7.61(1H, s), 7.49(1H, t), 3.74 (3H, s), 2.07 (3H, s)

m/e 346 (M−1⁺, 100%)

### EXAMPLE 10

### 2,3-Dichloro-N-[5-methoxy-2-trifluoromethyl-4-pyrimidinyl]benzenesulphonamide

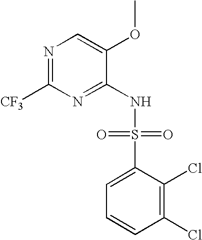

### a) 4-Chloro-5-methoxy-2-trifluoromethyl-pyrimidine

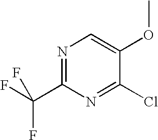

Ethyl formate (16.4 g) was added dropwise to a suspension of sodium methoxide (24.0 g) in dry tetrahydrofuran (150 mL) maintaining a temperature of 5-10° C. Methylmethoxy acetate (23 g) was then added dropwise maintaining a temperature of 5-10° C. After 2 h at room temperature, methanol (100 mL) was added followed by 2,2,2-trifluoromethylacetamidine (25 g) added dropwise maintaining a temperature below 30° C. The mixture was stirred at room temperature for 12 h then at 60° C. for 2 h. After cooling the pH was adjusted to 3 using conc. hydrochloric acid and 5-methoxy-2-trifluoromethyl-4-pyrimidinol collected as a white solid and dried. (10 g).

The 2-trifluoromethyl-4-pyrimidinol in phosporus oxychloride (100 mL) was heated under reflux for 16 h. and then evaporated under reduced pressure. The residue was added to ice/water and the white solid collected. Purification was by silica gel chromatography eluting with dichloromethane to give the subtitle compound (3 g).

¹H NMR (CDCl₃) δ 8.39(1H, s), 4.10 (3H, s)

### b) 2,3-Dichloro-N-[5-methoxy-2-trifluoromethyl-4-pyrimidinyl]benzenesulphonamide

Prepared using the method of example 7 from 4-Chloro-5-methoxy-2-trifluoromethyl-pyrimidine.

m/e 401 (M−1⁺, 100%)

MP 190-191° C.

¹H NMR (D6-DMSO) δ 8.44 (1H, s), 8.22 (1H, d), 7.97-7.96 (1H, dd), 7.62 (1H, t), 3.99 (3H, s)

### EXAMPLE 11

### 5-Chloro-thiophene-2-sulphonic acid, [2-chloro-5-methoxy-4-pyrimidinyl]amide

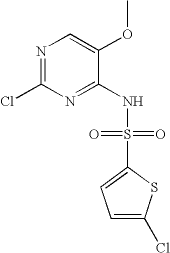

Prepared using the method of example 7 from 2,3-Dichloro-5-methoxy-pyrimidine and 5-chloro-thiophene-2-sulphonic acid amide

m/e 338 (M−1⁺, 100%)

MP 201-202° C.

¹H NMR (D6-DMSO) δ 8.19 (1H, s), 7.68 (1H, d), 7.27 (1H, d), 3.87 (3H, s)

### EXAMPLE 12

### 5-Chloro-thiophene-2-sulphonic acid, [5-methoxy-2-methyl-4-pyrimidinyl]amide

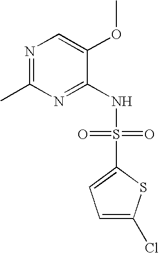

Prepared using the method of example 7 from 4-chloro-5-methoxy-2-methylpyrimidine and 5-chloro-thiophene-2-sulphonic acid amide

m/e 318 (M−1⁺, 100%)

MP 249-250° C.

¹H NMR (D6-DMSO) δ 7.65 (1H, s), 7.43 (1H, d), 7.07 (1H, d), 3.72 (3H, s), 2.41(3H, s)

### EXAMPLE 13

### 5-Chloro-N-[6-chloro-4-methoxy-3-pyridazinyl]thiophene-2-sulphonamide

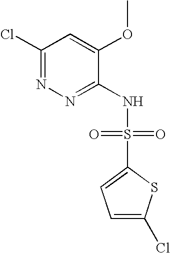

The title compound was prepared from 3-amino-6-chloro-4-methoxypyridazine (0.34 g), 5-chlorothiophene-2-sulphonyl chloride (0.46 g), 60% sodium hydride (0.17 g) and 1,2-dimethoxyethane (20 mL) by the method of Example 2. Purification was by chromatography on silica gel eluting with dichloromethane/iso-propanol mixtures and reverse phase preparative HPLC eluting with 0.1% aqueous ammonia/methanol mixtures. Gave 0.03g.

m/e 340 (M+1⁺, 100%)

¹H NMR (D6-DMSO) δ 7.51 (1H, s), 7.05-6.90 (2H, m), 3.81 (3H, s)

MP 120-122° C.

Pharmacological Data

FMAT Whole cell binding assay

Cells

CHO-K1 cells stably expressing the human recombinant CCR4 receptor (Euroscreen; Brussels, Belgium) were cultured in NUT.MIX.F_{—}12(HAM) medium with glutamax-1, containing 10% (v/v) foetal bovine serum and 400 μg ml⁻¹ geneticin.

Cells were harvested at approximately 70% confluence by treatment with a cell dissociation buffer, and seeded at 5×10³ cells/100 μl culture medium into wells of a black Costar clear-bottomed 96-well microtitre plates. Plates were incubated overnight at 37° C. in 5% CO₂ and used the following day.

Assay

Before use, the cell plates were washed twice with 100 μl Hanks balanced salt solution (HBSS). To each well was then added 65 μl of HBSS, 10 μL of 10% DMSO in HBSS±test compound and then 25 μL of 2.8 nM FB-MDC (Applied Biosystems). This fluorescent probe was prepared from a 10 μM stock in 0.08% (v/v) TFA/16% (v/v) acetonitrile, diluted into HBSS.

After two hours incubation in the dark at room temperature, the plates were analysed in an FMAT8100 reader (Applied Biosystems) to measure fluorescence that was associated with binding of FB-MDC to the cells. Compound activity was determined as an pIC₅₀ [log(concentration of compound that results in 50% inhibition)], comparing fluorescence in control and background wells.

Typical Data

All the compounds of the examples have a pIC₅₀ of greater than 5.0.

1. A compound of formula (I) and pharmaceutically acceptable salts or solvates thereof: 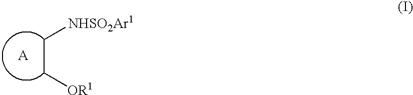
in which:
Ar¹ is dichlorophenyl or thienyl substituted by one or two chlorine atoms;
A is a pyrimidine, pyridazine or 1,2,4-triazine ring, each of which can be optionally substituted by one or more groups selected from hydroxyl, halogen, cyano, C₁₋₆alkyl, C₁₋₆alkoxy or C₃₋₆cycloalkyl where in each case the alkyl group may be substituted with 1-3 fluorine atoms, a cyano group or a hydroxy group;
R¹ is C₁₋₆alkyl or C₃₋₆cycloalkyl each of which can be optionally substituted with 1-3 fluorine atoms or a cyano group or R¹ is C₃₋₆alkenyl or C₃₋₆alkynyl or C₁₋₆alkyl-R²
R² is an aryl group or a 5-7 membered heteroaromatic ring containing 1-4 heteroatoms selected from nitrogen, oxygen or sulphur each of which can be optionally substituted by 1-3 groups selected from halogen, C₁₋₆alkyl, C₁₋₆alkoxy, ═O, ═S, CN or (CH₂)nOH where n is 1 or 2.

2. A compound according to claim 1 in which Ar¹ is 2,3-dichlorophenyl.

3. A compound according to claim 1 in which A is pyrimidine substituted by halogen or C₁₋₆alkoxy.

4. A compound according to claim 1 in which R¹ is C₁₋₆alkyl.

5. A compound according to claim 1 wherein when Ar¹ is dichlorophenyl A is pyrimidine or a 1,2,4-triazine ring.

6. A compound according to claim 1 which is selected from the group consisting of:
2,3-Dichloro-N-[4-methoxy-3-pyridazinyl]benzenesulphonamide
2,3-Dichloro-N-[6-chloro-4-methoxy-3-pyridazinyl]benzenesulphonamide.
2,3-Dichloro-N-[6-chloro-4-(3-pyridinylmethoxy)-3-pyridazinyl]benzenesulphonamide.
2,3-Dichloro-N-[3-chloro-6-methoxy-1,2,4-triazin-5-yl]benzenesulphonamide
2,3-Dichloro-N-[2,4-dimethoxy-5-pyrimidinyl]benzenesulphonamide
2,3-Dichloro-N-[4-methoxy-5-pyrimidinyl]benzenesulphonamide
2,3-Dichloro-N-[2-chloro-5-methoxy-4-pyrimidinyl]benzenesulphonamide
2,3-Dichloro-N-[5-methoxy-4-pyrimidinyl]benzenesulphonamide
2,3-Dichloro-N-[5-methoxy-2-methyl-4-pyrimidinyl]benzenesulphonamide
2,3-Dichloro-N-[5-methoxy-2-trifluoromethyl-4-pyrimidinyl]benzenesulphonamide
5-Chloro-thiophene-2-sulphonic acid, [2-chloro-5-methoxy-4-pyrimidinyl]amide
5-Chloro-thiophene-2-sulphonic acid, [5-methoxy-2-methyl-4-pyrimidinyl]amide and
5-Chloro-N-[6-chloro-4-methoxy-3-pyridazinyl]thiophene-2-sulphonamide
and pharmaceutically acceptable salts and solvates thereof.

7. A pharmaceutical composition comprising a compound of formula (I), or a pharmaceutically acceptable salt or solvate thereof, as claimed in claim 1 in association with a pharmaceutically acceptable adjuvant, diluent or carrier.

8-9. (canceled)

10. A method of antagonizing CCR4 in a patient, the method comprising administering to a patient a therapeutically effective amount a compound of formula (I), or a pharmaceutically acceptable salt or solvate thereof, as claimed in claim 1.

11. A method of treating a CCR4 mediated disease, the method comprising administering to a patient a therapeutically effective amount of a compound of formula (I).

12. A method of treating a chemokine mediated disease wherein the chemokine binds to one or more chemokine receptors, which comprises administering to a patient a therapeutically effective amount of a compound of formula (I), or a pharmaceutically acceptable salt or solvate thereof, as claimed in claim 1.

13. A method according to claim 12 in which the chemokine receptor belongs to the CCR chemokine receptor subfamily.

14. A method according to claim 12 in which the chemokine receptor is the CCR4 receptor.

15. A method of treating an inflammatory disease in a patient suffering from, or at risk of, said disease, which comprises administering to the patient a therapeutically effective amount of a compound of formula (I), or a pharmaceutically acceptable salt or solvate thereof, as claimed in claim 1.

16. A method according to claim 15 wherein the disease is asthma.

17. A process for the preparation of a compound of formula (I), claim 1 which comprises reacting a compound of formula (II): 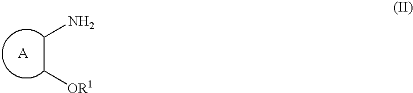
in which A and R¹ are as defined in formula (I) or are protected derivatives thereof, with a compound of formula (III):
Ar¹SO₂L (III)
in which Ar¹ is as defined in formula (I) or is a protected derivative thereof, and L is a leaving group,
and optionally thereafter:
removing any protecting groups
forming a pharmaceutically acceptable salt or solvate.
